SEQUENCE LISTING (3 sequences)

SEQ ID NO: 1 - DNA, 18 bp, artificial sequence
  synthetic oligonucleotide
taggaaggaaggagagtg

SEQ ID NO: 2 - DNA, 24 bp, artificial sequence
  synthetic oligonucleotide
cccaacaaaaataatattttaaca

SEQ ID NO: 3 - DNA, 22 bp, artificial sequence
  synthetic oligonucleotide
caaaaataatattttaacaacc

(19) United States
(12) Patent Application Publication
(10) Pub. No.: US 2013/0164279 A1
(43) Pub. Date: Jun. 27, 2013

(54) Micro RNA-148A as a Biomarker for Advanced Colorectal Cancer

(71) Applicant: BAYLOR RESEARCH INSTITUTE, Dallas, TX (US)

(72) Inventors: Ajay Goel, Dallas, TX (US); C. Richard Boland, Dallas, TX (US); Masanobu Takahashi, Richardson, TX (US)

(73) Assignee: BAYLOR RESEARCH INSTITUTE, Dallas, TX (US)

(21) Appl. No.: 13/708,537

(22) Filed: Dec. 7, 2012

Related U.S. Application Data
(60) Provisional application No. 61/579,321, filed on Dec. 22, 2011.

(51) Int. Cl. C12Q 1/68

(52) U.S. Cl. CPC C12Q 1/6886
USPC 424/133.1; 435/6.11; 424/158.1; 424/142.1

(57) ABSTRACT

The present invention includes methods of detection, diagnosis, prognosis, and treatment of a patient suspected of having a colorectal cancer comprising obtaining one or more samples of the patient, determining a level of expression of miR-148a or the level of methylation of a miR-148a promoter, and predicting a response to a cytotoxic chemotherapy cancer treatment.

### CROSS-REFERENCE TO RELATED APPLICATIONS

This application claims priority to U.S. Provisional Application Ser. No. 61/579,321, filed Dec. 22, 2011, the entire contents of which are incorporated herein by reference.

### STATEMENT OF FEDERALLY FUNDED RESEARCH

This invention was made with U.S. Government support under Contract Nos. R01 CA72851 and CA129286 awarded by the National Cancer Institute (NCI)/National Institutes of Health (NIH). The government has certain rights in this invention.

### TECHNICAL FIELD OF THE INVENTION

The present invention relates to the field of cancer detection, diagnosis, prognosis, and treatment, and more particularly, to methods involving the expression of miR-148a as a predictive or prognostic tool in the management of treatment for patients with advanced colorectal cancers (CRCs).

### INCORPORATION-BY-REFERENCE OF MATERIALS FILED ON COMPACT DISC

None.

### BACKGROUND OF THE INVENTION

Without limiting the scope of the invention, its background is described in connection with genetic markers and for prognosis of cancers and colorectal cancers.

U.S. Patent Application Publication No. 20110251098 filed by Showe, et al., (Nov. 6, 2009) describes diagnostic miRNA biomarkers and expression profiles consisting of multiple miRNA biomarkers in the peripheral blood lymphocytes of non-small cell lung cancer (NSCLC) and chronic obstructive pulmonary disease (COPD) patients, including miR-148.

U.S. Patent Application Publication No. 20110171646 filed by Schmittgen, et al., (Dec. 7, 2010) describes pancreas-enriched miRNAs (e.g., miR-148a) as those miRNAs with 10-fold or greater expression in the pancreas tissue compared to the mean of the other 21 tissues (including colon). The application describes analysis of pancreatic cancer. Importantly, this application teaches the up-regulation of miR-148a in pancreatic cancer.

U.S. Patent Application Publication No. 20100298151 filed by Taylor, et al., (Jul. 25, 2008) provides methods of diagnosis of ovarian cancer in a subject by measuring amounts of one or more microRNAs present in cancer-derived exosomes.

U.S. Patent Application Publication No. 20100197774 filed by Croce (Feb. 4, 2010), describes a method of diagnosing whether a subject has, or is at risk for developing, pancreatic cancer, comprising measuring the level of at least one miR gene product in a test sample from said subject, wherein an alteration in the level of the miR gene product in the test sample, relative to the level of a corresponding miR gene product (e.g., miR-148) in a control sample.

U.S. Patent Application Publication No. 20100113577 filed by Shi (Apr. 7, 2008), describes isolated nucleic acid molecule corresponding to miR-145 that are useful in treating colon cancer. The application also describes a method of diagnosing whether a subject has, or is at risk for developing, a cancer associated with low expression of miR-145 relative to normal in a subject, comprising: (1) reverse transcribing RNA from a test sample obtained from the subject to provide a target oligodeoxynucleotide; (2) hybridizing the target oligodeoxynucleotide to a miRNA-specific probe oligonucleotideto provide a hybridization profile for said test sample; and (3) comparing the test sample hybridization profile to a hybridization profile generated from a control sample, wherein an alteration in the signal is indicative of the subject either having, or being at risk for developing, the cancer.

U.S. Patent Application Publication No. 20090075258 filed by Latham, (Sep. 14, 2007), lists miR145 as oncomir (defined as a microRNA that is differentially expressed in at least one cancer or tumor-derived cell type). Regarding colorectal cancer, the application states that target miRNA may be selected from human miRNAs including but not limited to the let-7 family, but does not recite miR145. The patent application does not aim to evaluate the role of any specific miRNA and does not provide data on miR-148a as a possible biomarker for any disease.

Brandes, et al., “Identification by Real-time PCR of 13 mature microRNAs differentially expressed in colorectal cancer and non-tumoral tissues,” Molecular Cancer 2006, 5:29, describes miRNAs that whose expression were significantly altered in colorectal tumours compared to adjacent non-neoplastic tissues from patients and colorectal cancer cell lines. The publication states that miR-148a was overexpressed in CRC samples and colorectal cancer cell lines. Interestingly, Brandes teaches up-regulation of miR-148a expression.

Chen, et al., “Altered Expression of MiR-148a and MiR-152 in Gastrointestinal Cancers and Its Clinical Significance,” J. Gastrointestinal Surgery, Volume 14, Number 7, 1170-1179, states that expression levels of miR-148a and miR-152 in human gastric and colorectal cancers were significantly lower than that in their matched nontumor adjacent tissues. However, Chen did not identify a significant association between the miRNA expression status and prognosis or therapeutic benefit for patients.

### SUMMARY OF THE INVENTION

In one embodiment, the present invention includes a method to diagnose a stage of cancer of a patient suspected of having colorectal cancer comprising: obtaining a sample of the patient suspected of having colorectal cancer; determining a level of methylation of a miR-148a promoter; and diagnosing a stage of colorectal cancer if the expression of miR-148a is lower than in normal colonic tissue. In one aspect, the level of expression of miR-148a of a stage III tumor is significantly lower than those of normal colonic mucosa (0.104). In another aspect, the level of expression of miR-148a of a stage IV tumor is significantly lower than those of normal colonic mucosa (0.104). In another aspect, the level of expression of miR-148a of stage III (median, 0.080, P<0.001, Mann-Whitney U test) and IV tumors (0.077, P<0.001) is significantly lower than those of normal colonic mucosa (0.104). In another aspect, the step of determining the level of expression of miR-148a further comprises normalizing expression of miR-148a with expression of miR-16. In another aspect, the one or more samples are selected from the group consisting of a cancer biopsy, a tissue sample, a liver biopsy, a fecal sample, a cell homogenate, a blood, a serum, a plasma, one or more biological fluids, or any combinations thereof. In another aspect, the one or more samples comprise a cancer sample, a colorectal cancer sample, a control sample, or combinations thereof. In another aspect, the method further comprises the step of predicting a response to a cancer treatment comprises predicting that the patient will not benefit from cytotoxic chemotherapy if level of expression of miR-148a is less than the level in a normal sample. In another aspect, the step of predicting a response to a cancer treatment further comprises predicting that the patient will benefit from cytotoxic chemotherapy if level of expression of miR-148a is above the level in a normal sample. In another aspect, a low level of expression of miR-148a indicates at least one of reduced disease-free survival, progression-free survival (PFS), or overall survival (OS), of the patient if treated by cytotoxic chemotherapy cancer treatment. In another aspect, a low level of expression of miR-148a, indicates a reduced disease-free survival of the patient suspected of having stage II and III colon cancer if treated with a thymidylate synthase inhibitor. In another aspect, a low level of expression of miR-148a, indicates a reduced disease-free survival of the patient suspected of having stage II and III colon cancer if treated with 5-fluorouracil (5-FU) or analogs thereof. In another aspect, a low expression of miR-148a, indicates a reduced disease-free survival of the patient suspected of having stage IV colon cancer if treated with 5-FU and oxaliplatin-based chemotherapy.

Another embodiment of the present invention includes a method to manage a treatment of a patient suspected of having a colorectal cancer comprising: obtaining one or more samples of the patient; determining a level of expression of miR-148a; and predicting a response to a cytotoxic chemotherapy cancer treatment. In one aspect, the step of predicting a response to a cancer treatment further comprises predicting that the patient will not benefit from cytotoxic chemotherapy if level of expression of miR-148a is less than the level in a normal sample. In another aspect, the step of predicting a response to a cancer treatment further comprises predicting that the patient will benefit from cytotoxic chemotherapy if level of expression of miR-148a is above the level in a normal sample. In another aspect, a low level of expression of miR-148a indicates at least one of reduced disease-free survival, progression-free survival (PFS), or overall survival (OS), of the patient if treated by cytotoxic chemotherapy cancer treatment. In another aspect, a low level of expression of miR-148a, indicates a reduced disease-free survival of the patient suspected of having stage II and III colon cancer if treated with a thymidylate synthase inhibitor. In another aspect, a low level of expression of miR-148a, indicates a reduced disease-free survival of the patient suspected of having stage II and III colon cancer if treated with 5-fluorouracil (5-FU) or analogs thereof. In yet another aspect, a low expression of miR-148a, indicates a reduced disease-free survival of the patient suspected of having stage IV colon cancer if treated with 5-FU and oxaliplatin-based chemotherapy. In another aspect, the step of determining the level of expression of miR-148a further comprises normalizing expression of miR-148a with expression of miR-16. In another aspect, the one or more samples are selected from the group consisting of a cancer biopsy, a tissue sample, a liver biopsy, a fecal sample, a cell homogenate, a blood, a serum, a plasma, one or more biological fluids, or any combinations thereof. In another aspect, the one or more samples comprise a cancer sample, a colorectal cancer sample, a control sample, or combinations thereof. In another aspect, the step of predicting a response to a cancer treatment comprises predicting disease-free survival (DFS), progression-free survival (PFS), overall survival (OS), or combinations thereof. In another aspect, the step of predicting a response to a cancer treatment further comprises predicting a higher colorectal metastatic stage if expression of miR-148a is above the median for miR-148a expression in a normal tissue. In another aspect, the colorectal cancer is advanced colorectal cancer. In another aspect, the colorectal cancer is stage II, stage III, or stage IV. In another aspect, the method further comprises indicating cytotoxic chemotherapy if the level of expression of miR-148a is above the median for miR-148a expression in a normal tissue. In another aspect, the method further comprises contraindicating cytotoxic chemotherapy if the level of expression of miR-148a is below the median for miR-148a expression in a normal tissue.

Yet another embodiment of the present invention includes a method for selecting a cancer therapy for a patient diagnosed with metastatic colorectal cancer comprising the steps of: determining a level of expression of miR-148a in one or more biological samples of the patient; and selecting a first or second cancer therapy based on the level of expression of miR-148a; and treating the patient with a first cancer therapy comprising anti-growth hormone or anti-hormone receptor therapy or treating the patient with a second cancer therapy comprising cytotoxic chemotherapy. In one aspect, the anti-growth hormone comprises a VEGF antagonist, an anti-VEGF antibody, bevacizumab. In another aspect, the anti-growth hormone receptor comprises an EGFR antagonist, an anti-EGFR antibody, cetuximab, or panitumumab. In another aspect, the step of determining miR-148a activity further comprises comparing level of expression of miR-148a with a level of expression of a control. In another aspect, the one or more samples are selected from the group consisting of a cancer biopsy, a tissue sample, a liver biopsy, a fecal sample, a cell homogenate, a blood, a serum, a plasma, one or more biological fluids, or any combinations thereof. In another aspect, the one or more samples comprise a colorectal cancer sample, a control sample, or combinations thereof. In another aspect, the step of selecting survival of the patient further comprises selecting cytotoxic chemotherapy if miR-148a activity is high.

Yet another embodiment of the present invention includes a method to predict survival of a patient suspected of having colorectal cancer comprising: obtaining one or more biological samples of the patient; determining a level of expression of miR-148a; and predicting survival probability of the patient. In one aspect, the colorectal cancer is stage II or stage III and predicting survival probability comprises predicting a 5-year disease-free survival of less than 54% if the level of expression of miR-148a is below 0.69-fold of a level of expression of miR-148a of normal mucosa. In another aspect, the step of predicting survival of the patient further comprises predicting disease-free survival (DFS), progression-free survival (PFS), overall survival (OS), or combinations thereof. In another aspect, the step of predicting survival of the patient further comprises predicting a higher or lower colorectal metastatic stage. In another aspect, the colorectal cancer is advanced colorectal cancer. In another aspect, the colorectal cancer is stage II, stage III, or stage IV. In another aspect, the method further comprises treating the patient with a chemotherapy if the patient is predicted to benefit from cytotoxic cancer treatment. In another aspect, the chemotherapy comprises treatment with 5-fluorouracil or a combination of Folinic Acid (FOL), Fluorouracil (5-FU) and Oxaliplatin (OX), or irinotecan.

Yet another embodiment of the present invention includes a method of performing a clinical trial to evaluate a candidate drug believed to be useful in treating colorectal cancer, the method comprising: (a) determining a level of miR-148a expression in one or more biological sample of the patient; (b) administering a candidate drug to a first subset of patients, and a placebo to a second subset of patients; a comparable drug to a second subset of patients; or a drug combination of the candidate drug and another active agent to a second subset of patients; (c) repeating step (a) after the administration of the candidate drug or the placebo, the comparable drug or the drug combination; and (d) monitoring a change in the level of miR-148a expression of the first subset of patients as compared to the second subset of patients, wherein a statistically significant increase indicates that the candidate drug is useful in treating colorectal cancer.

Yet another embodiment of the present invention includes a method to diagnose a stage of cancer of a patient suspected of having colorectal cancer comprising: obtaining a sample of the patient suspected of having colorectal cancer; determining a level of expression of miR-148a; and diagnosing a stage of colorectal cancer, wherein the level of expression of miR-148a of stage III (median, 0.080, P<0.001, Mann-Whitney U test) and IV tumors (0.077, P<0.001) is significantly lower than those of normal colonic mucosa (0.104).

Yet another embodiment of the present invention includes a method to manage a treatment of a patient suspected of having a colorectal cancer comprising: obtaining one or more samples of the patient; determining a level of methylation of a miR-148a promoter; and predicting a response to a cytotoxic chemotherapy cancer treatment.

Yet another embodiment of the present invention includes a method for selecting a cancer therapy for a patient diagnosed with metastatic colorectal cancer comprising the steps of: determining a level of methylation of a miR-148a promoter in one or more biological samples of the patient; and selecting the cancer therapy based on the determination of the level of methylation of the miR-148a promoter; and treating the patient with a first treatment comprising an anti-growth hormone or anti-hormone receptor therapy; or treating the patient with a second treatment comprising cytotoxic chemotherapy.

Yet another embodiment of the present invention includes a method to predict survival of a patient suspected of having colorectal cancer comprising: obtaining one or more biological samples of the patient; determining a level of methylation of a miR-148a promoter; and predicting survival probability of the patient.

Yet another embodiment of the present invention includes a method of performing a clinical trial to evaluate a candidate drug believed to be useful in treating colorectal cancer, the method comprising: (a) determining a level of methylation of a miR-148a promoter in one or more biological samples of patients; (b) administering a candidate drug to a first subset of patients, and a placebo to a second subset of patients; a comparable drug to a second subset of patients; or a drug combination of the candidate drug and another active agent to a second subset of patients; (c) repeating step (a) after the administration of the candidate drug or the placebo, the comparable drug or the drug combination; and d) monitoring a change in the level of methylation of the miR-148a promoter of the first subset of patients as compared to the second subset of patients, wherein a statistically significant reduction indicates that the candidate drug is useful in treating colorectal cancer.

Yet another embodiment includes a method to diagnose a stage of cancer of a patient suspected of having colorectal cancer comprising: obtaining a sample from the patient suspected of having colorectal cancer; determining a level of methylation of a miR-148a promoter or the level of expression of miR-148a; and diagnosing a stage of colorectal cancer if the level of methylation of the miR-148a promoter is lower than in normal colonic tissue or the level of expression of miR-148a is higher than in normal colonic tissue.

Another embodiment is a method to manage a treatment of a patient suspected of having a colorectal cancer comprising: obtaining one or more samples of the patient; determining a level of expression of miR-148a; and predicting a response to a cytotoxic chemotherapy cancer treatment, wherein an increase in the level of expression of miR-148a is predictive of an increased responsiveness to the cytotoxic chemotherapy.

Yet another aspect of the present invention includes a kit for determining the stage of colorectal cancer in a human subject comprising: a biomarker detecting reagent for measuring level of methylation of a miR-148a promoter or the level of expression of miR-148a in a sample obtained from the human subject; and instructions for the use of the biomarker detecting reagent in determining the stage of colorectal cancer, wherein the instructions comprise providing step-by-step directions to compare the level of methylation of the miR-148a promoter or the level of expression of miR-148a from the sample, wherein a decrease in the methylation of the miR-148a promoter or an increase in expression of miR-148a in the sample versus a normal colonic tissue is indicative of a higher stage of colorectal cancer. In one aspect, the level of methylation of the miR-148a promoter is determined by quantitative bisulfite pyrosequencing, thin layer chromatography (TLC), high performance liquid chromatography (HPLC), mass spectrometry (MS), nanopore amperometry, nanopore sequencing, single-molecule, real-time (SM-RT) sequencing, endonuclease digestion, microarrays, matrix-assisted laser desorption ionization time-of-flight (MALDI-TOF) mass spectrometry, and next-generation sequencing. In another aspect, the biological samples are selected from the group consisting of a tissue sample, a plasma sample, a fecal sample, a cell homogenate, a blood sample, one or more biological fluids, or any combinations thereof. In another aspect, the level of expression of miR-148a from the sample is determined by nanostring, microarray expression profiling, PCR, reverse transcriptase PCR, reverse transcriptase real-time PCR, quantitative real-time PCR, end-point PCR, multiplex end-point PCR, cold PCR, ice-cold PCR, mass spectrometry, or nucleic acid sequencing. In another aspect, a low level of expression of miR-148a indicates at least one of reduced disease-free survival, progression-free survival (PFS), or overall survival (OS), of the patient if treated by cytotoxic chemotherapy cancer treatment.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D. MiR-148a expression and methylation in colonic mucosa from healthy individuals and in CRC tissues from patients. FIG. 1A. miR-148a expression in colonic mucosa from healthy controls (NC), and in stage II, III and IV CRCs; the number of patients (N) and median expression (Median) are listed below the graph. FIG. 1B. In situ hybridization for miR-148a in CRC tumors and normal mucosa, in which the chromogen stains red, and the counterstain blue. Representative photomicrographs are shown from a normal colonic mucosa (top panels), a tumor with low miR-148a expression (middle panels), and a tumor with high miR-148a expression (lower panels) at indicated magnifications. A photomicrograph is shown from a tumor with high miR-148a expression using a scramble probe as a negative control (bottom, left panel). FIG. 1C. miR-148a methylation levels in stage IV tumors. The putative promoter region of miR-148a, and the position of pyrosequencing primers are illustrated in the top panel. The scatter plot of miR-148a expression and methylation levels are shown in the bottom panel. FIG. 1D. miR-148a expression levels are shown for methylated and nonmethylated CRCs in the top panel. miR-148a methylation levels are shown for tumors with high and low miR-148a expression in the bottom panel. One outlier value (the methylation level; 48%) is excluded from the methylated group in the bottom graph.

FIGS. 2A to 2D show survival analysis in stage II/III patients treated with 5-Fluorouracil (5-FU)-based adjuvant chemotherapy. Kaplan-Meyer curves for disease-free survival (DFS; FIG. 2A) and overall survival (OS; FIG. 2B) in stage II/III patients according to miR-148a expression. Kaplan-Meyer curves for OS in stage II (FIG. 2C) and stage III (FIG. 2D) according to miR-148a expression.

FIGS. 3A-3E demonstrate the correlation between miR-148a status and therapeutic response or survival in stage IV patients treated with 5-FU and oxaliplatin. FIG. 3A shows the chemotherapeutic response according to miR-148a expression. Complete response, CR; partial response, PR; stable disease, SD; progressive disease; PD. Kaplan-Meyer curves for progression-free survival (PFS; FIG. 3B) and OS (FIG. 3C) in stage IV patients according to miR-148a expression. Kaplan-Meyer curves for PFS (FIG. 3D) and OS (FIG. 3E) in stage IV patients according to miR-148a methylation.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum).

As used herein, the term “tissue sample” (the term “tissue” is used interchangeably with the term “tissue sample”) includes any material composed of one or more cells, either individual or in complex with any matrix obtained from a patient. The definition includes any biological or organic material and any cellular subportion, product or by-product thereof. The definition of “tissue sample” should be understood to include without limitation colorectal tissue samples, tissues suspected of including colorectal cancer cells, blood components, and even fecal matter or fluids that includes colorectal cells. Also included within the definition of “tissue” for purposes of this invention are certain defined acellular structures such as dermal layers of epithelium that have a cellular origin but are no longer characterized as cellular. The term “stool” or “feces” as used herein is a clinical term that refers to feces obtained from a mammal such as a human.

As used herein, the term “biological fluid” refers to a fluid containing cells and compounds of biological origin, and may include blood, stool or feces, lymph, urine, serum, pus, saliva, seminal fluid, tears, urine, bladder washings, colon washings, sputum or fluids from the respiratory, alimentary, circulatory, or other body systems. For the purposes of the present invention the “biological fluids”, the nucleic acids containing the biomarkers may be present in a circulating cell or may be present in cell-free circulating DNA or RNA.

As used herein, the term “gene” refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used herein, a “biomarker” refers to a molecular indicator that is associated with a particular pathological or physiological state. The “biomarker” as used herein is a molecular indicator for cancer, more specifically an indicator for distant metastasis of primary CRCs. Examples of “biomarkers” include miR-148a.

As used herein, the term “statistically significant” refers to differences between the groups studied, relates to condition when using the appropriate statistical analysis (e.g. Chi-square test, t-test) the probability of the groups being the same is less than 5%, e.g. p<0.05. In other words, the probability of obtaining the same results on a completely random basis is less than 5 out of 100 attempts. The skilled artisan will recognize that there will be variability in certain measurements, for example, the level of mir-148a expression was determined by normalizing the expression to, e.g., miR-16, thus, the number 0.069-fold is not a definitive number. As a general matter, when the terms “higher” or “lower” are used to indicate the level of expression of a MiR, this indicates a statistically “higher” or “lower” level of expression for that same marker (e.g., miR-148a) in a CRC sample versus normal mucosa. As demonstrated in the figures disclosed herein (where expression is generally shown as a range), the skilled artisan can determine the statistical significance of the measured biomarker in relation to that expressed in normal colorectal tissue from the same patient. Thus, the cut-off value can be determined in the context of the same patient, thus yielding a statistically significant measurement for an increase or decrease in expression. It is also possible to measure invariant markers from CRC, e.g., miR-16, that can also be used to normalize levels of expression.

As used herein, the term “kit” or “testing kit” denotes combinations of reagents and adjuvants required for an analysis. Although a test kit consists in most cases of several units, one-piece analysis elements are also available, which must likewise be regarded as testing kits.

The level of methylation of the miR-148a promoter can be determined by well-known methods. For example, the level of methylation can be determined by a number of mehods, including but not limited to: quantitative bisulfite pyrosequencing, thin layer chromatography (TLC), high performance liquid chromatography (HPLC), mass spectrometry (MS), nanopore amperometry, nanopore sequencing, single-molecule, real-time (SM-RT) sequencing, endonuclease digestion, microarrays, matrix-assisted laser desorption ionization time-of-flight (MALDI-TOF) mass spectrometry, and next-generation sequencing. The level of expression of miR-148a from a sample can be determined by any number of well-known methods, including but not limited to: nanostring, microarray expression profiling, PCR, reverse transcriptase PCR, reverse transcriptase real-time PCR, quantitative real-time PCR, end-point PCR, multiplex end-point PCR, cold PCR, ice-cold PCR, mass spectrometry, or nucleic acid sequencing.

Colorectal cancer (CRC) is still the second leading cause of cancer-related deaths in the United States (1). In spite of the improved screening modalities for earlier detection in recent years a significant proportion of individuals are diagnosed with advanced stage CRC. Currently, patients with colon cancer with lymph node metastasis (TNM stage III) are treated with adjuvant chemotherapy by cytotoxic drugs such as 5-fluorouracil (5-FU) and oxaliplatin, following surgical resection of their cancer, to reduce the risk of tumor recurrence. Patients with locally advanced or distant metastatic CRC (stage IV) are treated with the combinations of such chemotherapeutic drugs and molecular-targeted drugs (anti-VEGF and anti-EGFR antibodies). Although these drug therapies have improved survival of such patients, a significant proportion of them receive chemotherapy with no benefit or even worse outcome because of their toxicities. The present inventors recognize that it has been required to develop robust biomarkers that identify who will or will not benefit from such drug therapies.

The mutation status of KRAS gene is an established predictive marker for selecting treatment strategies in CRC. Patients with tumors harboring a mutation in codon 12 or 13 in this gene do not benefit from anti-EGFR-based drug therapy (2, 3) and the screening for this mutational status is recommended for all of patients with stage IV disease who are considered to receive anti-EGFR antibody-based drug therapy (the National Comprehensive Cancer Network guideline: www.nccn.org).

As used herein, the “Stage” of the colorectal cancer refers to the standard TNM system (T is the size of the tumor and whether it has invaded nearby tissue, N is the extent to which regional lymph nodes are involved, and M distant metastasis) developed and maintained by the International Union Against Cancer (UICC) and followed by other organizations such as the American Joint Committee on Cancer (AJCC) and the International Federation of Gynecology and Obstetrics (FIGO).

The present inventors recognized that there are no established biomarkers for predicting therapeutic outcome of patients with stage III or IV CRC from conventional cytotoxic chemotherapy. Previously, the present inventors identified one predictive markers, a microsatellite instability (MSI) phenotype, which is present in ˜15% of CRC and characterized by instability of short nucleotide repeats in DNA sequence (4). The MSI phenotype is associated with favorable survival at least in stage II patients regardless of adjuvant chemotherapy, and with decreased benefit from 5-FU-based adjuvant chemotherapy in those patients (5, 6). The present inventors recognize that it is still uncertain whether MSI phenotype has any predictive value in stage III patients treated with adjuvant chemotherapy, given several conflicting reports (7). The present inventors recognized that inconsistent results could be attributed to the heterogeneity among MSI tumors including the existence of germline mutations in mismatch repair genes or hypermethylation of MLH1 (8). The present inventors also recognize that the data suggested that other molecular markers including a CpG island methylator phenotype, genome-wide gene expression profiling, or the status of specific genes (such as polymorphism, gene expression status, and protein status) involved in the repair of DNA damaged by cytotoxic drugs or in the drug metabolism (such as ERCC1, DPD and TS) have a potential as prognostic/predictive marker (9-12); however, other data demonstrated opposing results.

The present inventors found that dysregulation of microRNA (miRNA), small non-coding RNA of ˜22 nucleotides, is involved in early tumorigenesis as well as disease progression among various malignancies including CRC (13, 14). Each miRNA exerts its oncogenic and/or tumor suppressive functions mainly through their binding ability to the 3′-untranslated regions of gene transcripts, resulting in their suppression of translation or degradation. In CRC, for instance, miRNAs including miR-17˜92 family, miR-21, miR-31, miR-34b/c, miR-143, miR-145, and miR-203 were found to be dysregulated (15-18). Based upon the crucial involvement of alterations of miRNA in carcinogenesis, the present inventors recognized the need to identify specific miRNA(s) that predict(s) prognosis or therapeutic outcome in patients with various cancers; however, very few reports have demonstrated the potential of miRNA(s) as prognostic/predictive marker(s) in CRC. Although Schetter, et al., conducted a study demonstrating that miR-21 may be a promising prognostic/predictive marker in stage II/III CRC treated with 5-FU-based adjuvant chemotherapy, the number of patients treated with chemotherapy (stage II, N=14, and stage III, N=41) was relatively small (18). It has also been shown that patients with stage II CRC with high expression of miR-320 or miR-498 had better recurrence-free survival; however, the patient number was relatively small (N=37) as well (19). In addition, miRNAs as prognostic/predictive markers have not been validated yet in other larger studies. Finally, no report has demonstrated the possible potential of miRNA as predictive markers in stage IV CRC.

The present inventors recognized that miR-148a is one putative tumor suppressive miRNA involved in colorectal carcinogenesis (20, 21); and that miR-148a exerts a tumor suppressive function by targeting several oncogenic genes such as PXR, TGIF2, MSX1, CDC25B, DNMT1 and DNMT3b using other cell lines model (20, 22-26). The present inventors demonstrate herein that miR-148a expression status is useful not only for predicting prognosis and/or therapeutic outcome of patients with CRC, but to help make the decision of which treatment to pursue.

The present inventors have found that miR-148a is frequently down-regulated, in-part, through its promoter methylation in primary cancer tissues from a large cohort including 273 CRC patients. The miR-148a expression status was significantly correlated and associated with prognosis of patients with stage III colon cancer treated with 5-FU-based chemotherapy, and with therapeutic response and prognosis of those with stage IV CRC treated with 5-FU and oxaliplatin.

Conventional cytotoxic drug-based therapy remains the mainstay for the management of patients with advanced stage CRC. The present inventors found that miR-148a status is a predictive marker in CRC patients treated with chemotherapy.

RNA was extracted from 273 formalin-fixed paraffin-embedded primary CRC tissues of stage II and III patients treated with 5-fluorouracil-based (5-FU-based) adjuvant chemotherapy and stage IV patients treated with 5-FU and oxaliplatin-based chemotherapy. Taqman real-time RT-PCRs was performed to quantify the expression of miR-148a. In addition, the miR-148a promoter methylation levels were measured by pyrosequencing. The correlation between the miR-148a status and survival was analyzed and documented.

It was found that miR-148a expression was significantly down-regulated in advanced stage CRC compared to normal colonic mucosa. The low expression group had significantly shorter disease free-survival than the high expression group in stage III (P=0.007). The low expression group had significantly worse response rate (P=0.005) and poorer OS(P=0.024) in stage IV. The methylation status of miR-148a was correlated with its expression and was also associated with survival in stage IV patients. In multivariable Cox proportional-hazard model, miR-148a expression was an independent predictive marker in advanced CRC patients. These data show that the miR-148a status has a predictive impact in advanced CRC patients treated with chemotherapy.

It was also found that miR-148a expression status is an independent prognostic/predictive marker in stage III and IV colorectal cancer. Using a large cohort of 273 patients, it was demonstrated that miR-148a expression status was significantly associated with disease-free survival in stage II and III (especially in stage III) and with therapeutic response and survival in stage IV. In addition, miR-148a methylation was also associated with worse outcome in stage IV patients.

Formalin-fixed paraffin-embedded tissues of primary CRC from a cohort of 273 patients with CRC (76 of stage II and 125 of stage III colon cancer, and 72 of stage IV CRC) and those of normal colonic mucosa from 20 healthy individuals were obtained from the pathology Department of the Hospital Clinic of Barcelona, Spain. All of stage II (without lymph node metastasis) and III patients were treated with 5-FU-based adjuvant chemotherapy for 6 months following the resection of primary tumors, and all of stage IV patients were treated with 5-FU and oxaliplatin-based chemotherapy until the treatment failure. The chemotherapeutic response in stage IV patients was evaluated according to the Response Evaluation Criteria In Solid Tumors (RECIST) guideline (27). All individuals provided the written informed consent, and this study was approved by the Institutional Review Board of the hospital. The patients included in this study were enrolled between 1996 and 2008. All stage II and III patients were treated with 5-FU-based adjuvant chemotherapy for 6 months subsequent to tumor resection, and all stage IV patients were treated with 5-FU and oxaliplatin until the treatment failed. The stage II and III patients were followed-up every three months for the first two years, and every six months for the subsequent three years. Both locoregional relapse and/or distant metastasis were defined as tumor recurrence, whereas metachronous colorectal lesions were not considered as recurrence. The median follow-up times are 52.2 months (range; 2.9-173 months) in stage II and III patients, and 19.1 months (range; 3.7-83.7 months) in stage IV patients. Among stage II and III patients, 70 patients (35%) had tumor recurrence (median; 17.8 months, range: 5.5-144 months), and the median DFS of non-recurrence patients were 40.5 months (range; 7.5-155 months). The follow-up of patients was finished in November, 2009. Chemotherapeutic response in stage IV patients was evaluated according to the Response Evaluation Criteria In Solid Tumors (RECIST) guidelines [19] every two months. MSI status of tumors was determined by analyzing five mononucleotide markers (BAT-25, BAT-26, MONO-27, NR-21, and NR-24; MSI Analysis System, Promega, Madison, Wis., USA). The clinicopathological characteristics of the patients are shown in Table 1.

DNA and RNA extraction. DNA was extracted from 10 μm-thick formalin-fixed paraffin-embedded tissues with the QIAmp DNA FFPE tissue kit (Qiagen, Valencia, Calif.) according to the manufacturer's protocol. Total RNA including miRNA fraction was extracted from 10 μm-thick formalin-fixed paraffin-embedded tissues with the RecoverAll Total Nucleic Acid Isolation Kit (Ambion, Inc., Austin, Tex.) according to the manufacturer's protocol.

Multiplex quantitative RT-PCR. In a screening set that included normal colonic mucosa from healthy subjects and 44 CRC tissues (16 stage II and III each and 12 stage IV patients), the expression status of 21 candidate miRNAs (miR-9, miR-10b, miR-19a, miR-21, miR-31, miR-34a, miR-34c, miR-101, miR-103, miR-137, miR-143, miR-145, miR-148a, miR-148b, miR-152, miR-155, miR-194, miR-320, miR-335, miR-373 and miR-519c) was quantified using the high-throughput Fluidigm microfluidics dynamic arrays. Each Taqman miRNA assay (part no. 4427975, Applied Biosystems, Foster City, Calif., USA) was used in the multiplex RT-PCR analysis as follows: assay ID, 000583, 002218, 000395, 000397, 002279, 000426, 000428, 002253, 000439, 001129, 002249, 002278, 000470, 000471, 000475, 002623, 000493, 002277, 000546, 000561, and 001163, respectively. These candidate miRNAs have previously been shown to be involved in CRC and/or other human malignancies.

Quantification of miRNA expression by real-time RT-PCR. The expressions of miRNAs were quantified in the Taqman real-time reverse transcription-PCR (RT-PCR) following the manufacturer's protocol (Applied Biosystems, Foster city, CA) in the ABI 7000 sequence detection system (Applied Biosystems). In brief, 20 ng of total RNA from the FFPE tissues was reverse-transcribed and 6 ng of cDNA was used in each well for real-time RT-PCR. The following PCR cycle conditions were used: initial denaturation at 95° C. for 10 min, followed by 45 cycles at 95° C. for 15 sec, and 60° C. for 30 sec. Each reaction was performed in duplicate or triplicate. The expression level of miR-148a was calculated by delta Ct value to that of miR-16 (the difference between the Ct value of miR-148a and that of miR-16 as a reference). To keep the consistency throughout all plates, three independent RNA samples were loaded as internal controls in every run of PCR, and all results of plate were normalized according to the data of internal controls.

DNA methylation analysis. DNA was bisulfite modified according to manufacturer's protocol (EZ DNA methylation Gold Kit, Zymo Research, Irvine, Calif.). The methylation level of miR-148a promoter region was analyzed by pyrosequencing according to the manufacturer's protocol (PSQ HS 96A pyrosequencing system, Qiagen). The following primers were used; miR-148a forward primer, 5′-biotin-TAGGAAGGAAGGAGAGTG (SEQ ID NO: 1) miR-148a reverse primer, 5′-CCCAACAAAAATAATATTTTAACA (SEQ ID NO: 2), and miR-148a sequencing primer, 5′-CAAAAATAATATTTTAACAACC (SEQ ID NO: 3). The following PCR cycle conditions were used: initial denaturation at 94° C. for 7 min, followed by 45 cycles at 94° C. for 30 sec, 52° C. for 30 sec, and 72° C. for 30 sec.

In situ hybridization for miR-148a was performed with probes for miR-148a, RNU6b, and a scramble (Exiqon, Madrid, Spain). A fluorescein (FITC) 59-labeled locked nucleic acid-incorporated miRNA probe (miRCURY LNA detection probe, Exiqon, Woburn, Mass., USA) was used for visualization of miR-148a on 3 mm-thick FFPE tissue sections. A scrambled and an RNU6b probe were included as negative and positive controls, respectively (Exiqon). The slides were placed in an oven at 59° C. overnight. Sections were deparaffinized with xylene, rehydrated with ethanol, and treated with diethylpyrocarbonate water for 1 min. Chromogenic ISH was performed in an automated platform Bond Max (Vision BioSystems, Norwell, Massachusetts, USA). Slides were pretreated with protease 1 for 4 min at 37° C. A total of 300 ml 25-nM probe was hybridized in sodium chloride, sodium citrate hybridization buffer at 45° C. overnight. Immunologic detection was performed with a mouse anti-FITC antibody at 37° C. for 60 min followed by a biotin-free, polymeric horseradish peroxidase linker antibody conjugate system (Refine Detection System, Vision Bio Systems). DAB was used as the chromogen and hematoxylin was used as a counterstain.

Statistical analyses were performed with GraphPad Prism 4.0 (GraphPad Software, La Jolla, Calif., USA) or MedCalc v12 (MedCalc software, Belgium). The differences between two groups were analyzed by the Mann-Whitney U-test. Correlation analyses were carried out using Spearman's rank correlation method. The CRC tumors were categorized into high and low miR-148 expression groups using Receiver Operating Characteristic curve analysis (stage II/III) or the median expression values (stage IV). Kaplan-Meier analysis was performed to estimate the distributions of disease-free survival (DFS) and cancer-specific overall survival (OS) in stage II and III patients, and progression-free survival (PFS) and OS in stage IV patients. A log-rank test was used to analyze the statistical differences in survival as deduced from Kaplan-Meier curves. Cox proportional-hazard regression analysis was performed to calculate HR and 95% CI for each covariable. The final multivariate model was based upon a stepwise method for clinical factors associated with good or poor survival (p<0.1) in univariate models. For the survival analysis, the solitary MSI tumor was excluded from the stage IV group. All differences were regarded as statistically significant when p<0.05.

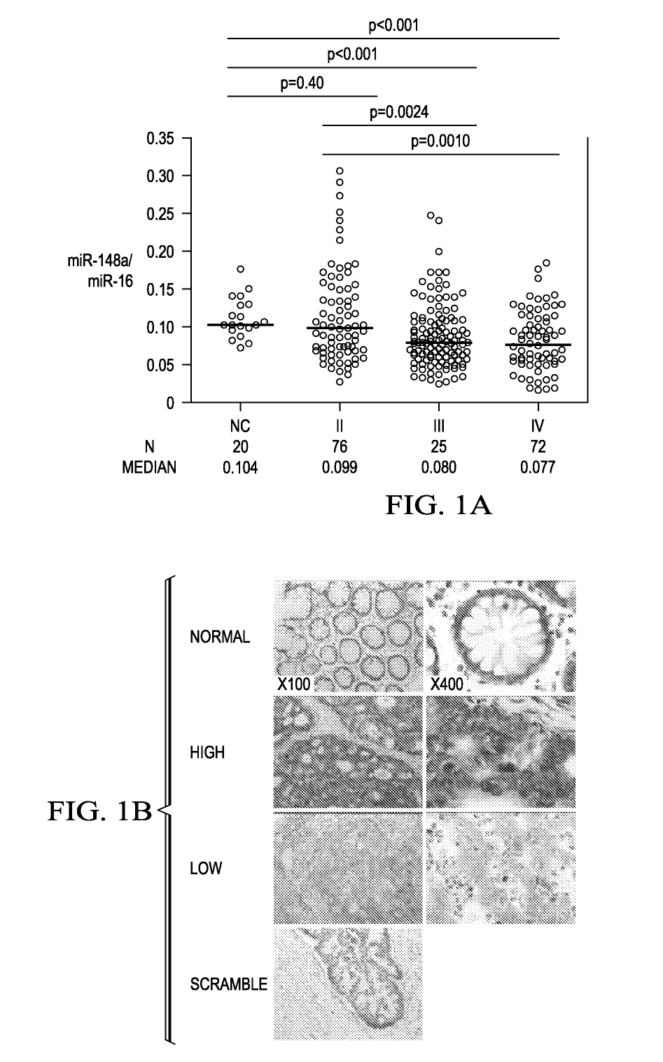

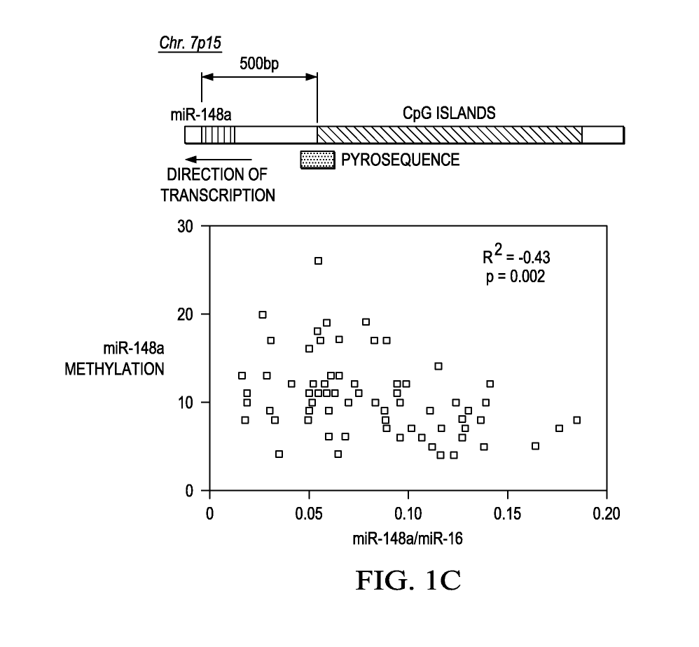

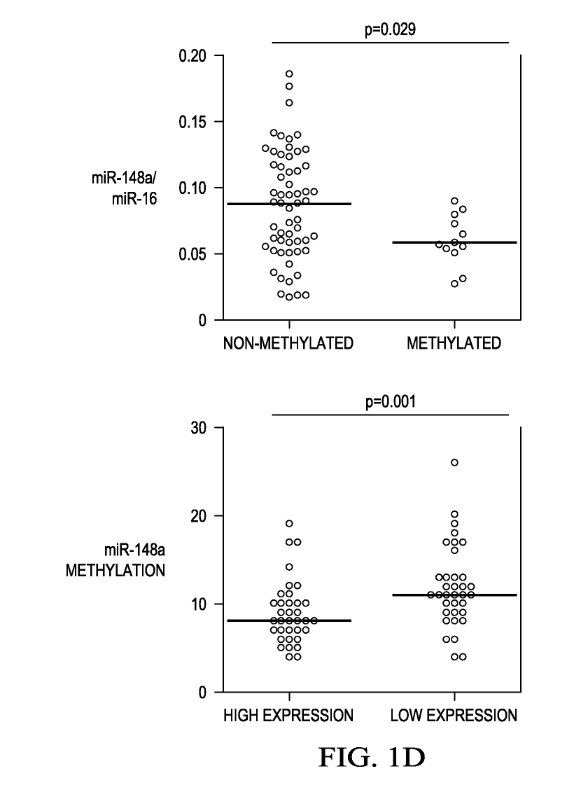

In our entire cohort (n=273), miR-148a expression in stage III/IV tumors was significantly lower than in normal colonic mucosa (p<0.001; FIG. 1A). We also observed a trend toward gradual lowering of miR-148a expression with advancing stage of the CRCs (FIG. 1A). More specifically, miR-148a expression in stage III and IV tumors was significantly lower than in normal colonic mucosa (p<0.001), while it was not significantly different between stage II tumors and the normal mucosal specimens (p=0.41; FIG. 1A).

To confirm the tumor-specific expression pattern for miR-148a, ISH analysis was performed in a subset of stage IV tumors with high and low miR-148a expression. It was observed that expression in normal colonic mucosa of stage II tumors was high, confirming the qRT-PCR results (FIG. 1B). CRCs with high miR-148a expression at qRT-PCR also expressed this miRNA primarily within the cytoplasm of neoplastic cells (FIG. 1B), but not in the non-epithelial stromal cells, except for the staining of some inflammatory cells in the lamina propria, particularly the plasma cells. Furthermore, CRCs with low miR-148a expression ascertained by qRT-PCR also revealed very low or absent expression of this miRNA at the ISH level (FIG. 1B). These results indicate that our qRT-PCR results accurately reflected the endogenous expression of miR-148a within the cancer cells obtained from CRC tissue specimens.

Expression of miR-148a is inversely correlated with its promoter methylation status. The present inventors recognize that the putative promoter region of miR-148a has CpG islands and its methylation is implicated in CRC and breast cancers (20, 28, 29). The present inventors appreciated for the first time the novelty of the correlation between expression and methylation status of this miRNA in a large cohort of patients. The present inventors elucidated that a miR-148a methylation-expression relationship and correlation exists in the present cohort of patients with CRC. Methylation analysis was focused on the stage IV cohort because the miR-148a expression was most down regulated in stage IV tumors and the existence of miR-148a methylation was observed most frequently in stage IV. As a result, quantitative pyrosequencing analyses showed that the miR-148a methylation was detected in relatively low level in stage IV tumors (median, 10%, ranged 4-26%), but when the methylation status was compared with the expression status, a significant correlation was observed (Spearman's coefficient, R²=−0.43, P<0.001; FIG. 1C). After categorizing categorized all tumors into a non-methylated (methylation level <15%) and methylated groups (15% methylation), it was observed that the methylated tumors had consistently lower miR-148a expression (0.068 vs. 0.088, p=0.029; FIG. 1D, top). Furthermore, CRCs with lower miR-148a expression were more frequently methylated compared to tumors with higher expression (median, 11% vs. 8%, p=0.001, Mann-Whitney U test; FIG. 1D, bottom). These results highlight that the hypermethylation of the putative miR-148a promoter region is an important regulatory mechanism for its expression in CRC.

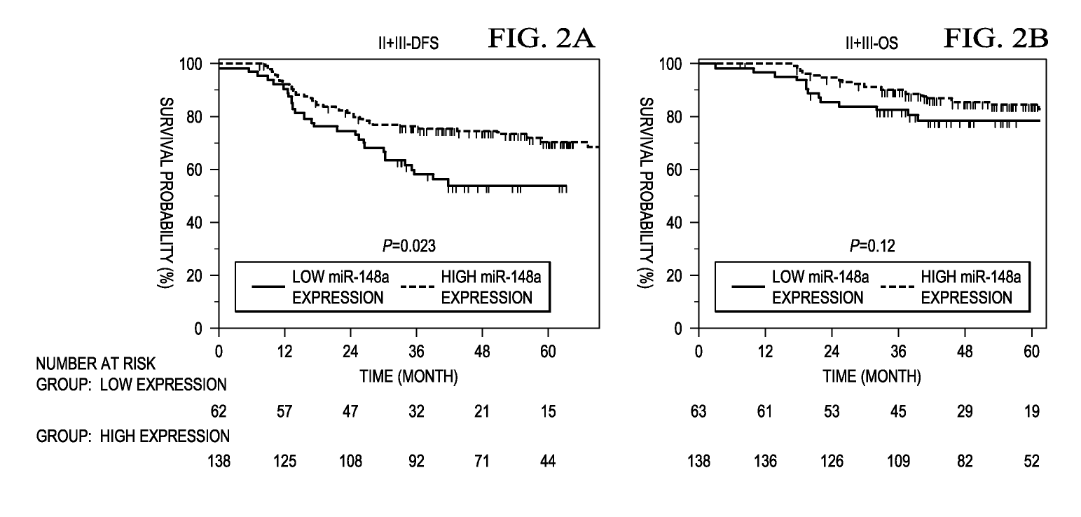

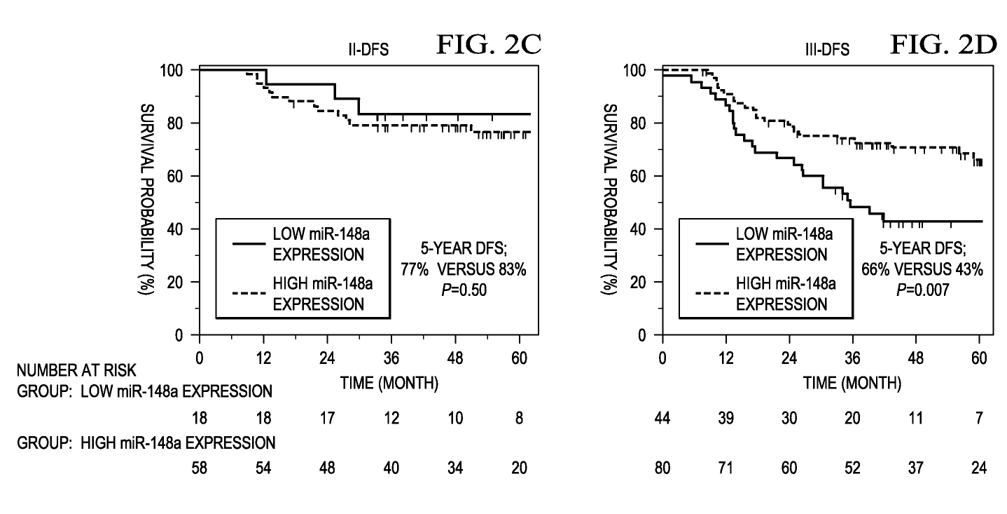

Low miR-148a expression is associated with poor outcome in patients with stage II and III CRC We next aimed to determine whether miR-148a expression status had an impact on prognosis in patients with stage II and III CRC treated with 5-FU-based adjuvant chemotherapy. For these analyses, the inventors compared the differences in DFS and OS between the high expression (stage II=58, III=80) and low expression (II=18, III=45) groups. The inventors did not find significant associations between the miR-148a expression and any of the clinicopathological factors such as age, gender, tumor location or MSI status (Table 1). However, low miR-148a expression was significantly associated with shorter DFS (5-year DFS, low vs. high, 54% vs. 71%, p=0.023; FIG. 2A), and showed a trend toward worse OS (5-year OS, 78% vs. 85%, p=0.12; FIG. 2B). Next, the inventors evaluated the prognostic/predictive value of miR-148a expression in a Cox proportional hazard regression model. In univariate analysis, higher TNM stage (III vs. II, HR 2.06, 95% CI 1.21-3.52, p=0.008) and lower miR-148a expression (HR 1.74, 95% CI 1.08-2.83, p=0.025) were significantly associated with shorter DFS, and younger age showed a trend towards shorter DFS (<60, HR 1.57, 95% CI 0.97-2.56, p=0.071; Table 2). Furthermore, in the multivariate model including these three factors, miR-148a expression status was independently associated with worse survival (HR 1.83, 95% CI 1.12-2.99, p=0.017; Table 2).

TABLE 1
miR-148 expression status and clinicopathologic characteristics of patients and tumors.

| | | Stage II + III | | | | | Stage IV | | | | |
|---|---|---|---|---|---|---|---|---|---|---|---|
| | | High expression | | Low expression | | | High expression | | Low Expression | | |
| | | (n = 138) | | (n = 63) | | | (n = 36) | | (n = 36) | | |
| | | N | % | N | % | P | N | % | N | % | P |
| Age, years | Median | 66.5 | | 68.5 | | 0.13^{a} | 58.5 | | 62.0 | | 0.36^{a} |
| | Range | 32-82 | | 45-82 | | | 43-78 | | 36-79 | | |
| Gender | Male | 79 | 57 | 39 | 62 | 0.64^{b} | 25 | 69 | 22 | 61 | 0.62^{b} |
| | Female | 59 | 43 | 24 | 38 | | 11 | 31 | 14 | 39 | |
| Tumors | Proximal | 39 | 28 | 22 | 35 | 0.41^{d} | 10 | 28 | 5 | 14 | 0.35^{d} |
| location^{c} | Distal | 99 | 72 | 41 | 65 | | 20 | 56 | 24 | 67 | |
| | Rectum | 0 | 0 | 0 | 0 | | 6 | 17 | 7 | 19 | |
| MSI | yes | 8 | 6 | 6 | 10 | 0.38^{b} | 0 | 0 | 1 | 3 | 1.00^{b} |
| | no | 130 | 94 | 57 | 90 | | 36 | 100 | 35 | 97 | |
| Performance | 0-1 | | | | | | 32 | 89 | 31 | 86 | 1.00^{b} |
| status | 2 | | | | | | 4 | 11 | 5 | 14 | |

^{a}The difference was analyzed by Mann-Whitney U test.
^{b}The difference was analyzed by Fisher's exact test.
^{c}Proximal colon, located above splenic flexure; distal colon, located in splenic flexure or below
^{d}The difference was analyzed by Chi-square test.

We next analyzed data from stage II and III CRC separately to determine whether the association between low miR-148a expression and worse outcome was uniform across both stages, or predominantly aligned with one stage. It was found that in stage II, miR-148a expression did not associate with DFS (5-year DFS, high vs. low, 77% vs. 83%, p=0.50; FIG. 2C) or OS (5-year OS, 89% vs. 87%, p=0.94; data not shown). However, in stage III, low miR-148a expression was significantly associated with poorer DFS (5-year DFS, 43% vs. 66%, p=0.0071; FIG. 2D) but not with OS (5-year OS, 75% vs. 81%, p=0.16; data not shown). Moreover, low miR-148a expression was an only factor that associated with tumor recurrence in stage III (Table 2). These results suggest that miR-148a expression status acts as a prognostic/predictive biomarker for stage III CRC.

TABLE 2
Univariable and multivariable analysis of miR-148a expression and disease-free survival in II/III CRC patients.

| | | II + III | | | | III only | | | |
|---|---|---|---|---|---|---|---|---|---|
| | | Univariable | | Multivariable | | Univariable | | Multivariable | |
| Variables | | HR (95% CI) | P | HR (95% CI) | P | HR (95% CI) | P | HR (95% CI) | P |
| Age, years | >60 | 1.0 | | 1.0 | | 1.0 | | | |
| | <60 | 1.57 (0.97-2.56) | 0.071 | 1.78 (1.09-2.92) | 0.022^{b} | 1.42 (0.79-2.56) | 0.24 | | |
| Gender | Male | 1.0 | | | | 1.0 | | | |
| | Female | 0.97 (0.60-1.57) | 0.91 | | | 0.89 (0.50-1.60) | 0.71 | | |
| Tumors | Proximal | 1.0 | | | | 1.0 | | | |
| location^{a} | Distal | 0.92 (0.55-1.51) | 0.73 | | | 1.15 (0.64-2.07) | 0.64 | | |
| MSI | No | 1.0 | | | | 1.0 | | | |
| | Yes | 0.78 (0.29-2.13) | 0.63 | | | 1.35 (0.49-3.73) | 0.57 | | |
| TNM stage | II | 1.0 | | 1.0 | | NA | | | |
| | III | 2.06 (1.21-3.52) | 0.008^{b} | 2.06 (1.20-3.53) | 0.009^{b} | NA | | | |
| Expression | High | 1.0 | | 1.0 | | 1.0 | | 1.0 | |
| | Low | 1.74 (1.08-2.83) | 0.025^{b} | 1.83 (1.12-2.99) | 0.017^{b} | 2.11 (1.21-3.68) | 0.009^{b} | 2.11 (1.21-3.68) | 0.009^{b} |

^{a}Proximal colon, located above splenic flexure; distal colon, located in splenic flexure or below
^{b}P < 0.05
Abbreviation: HR, hazard ratio; CI, confidence interval.

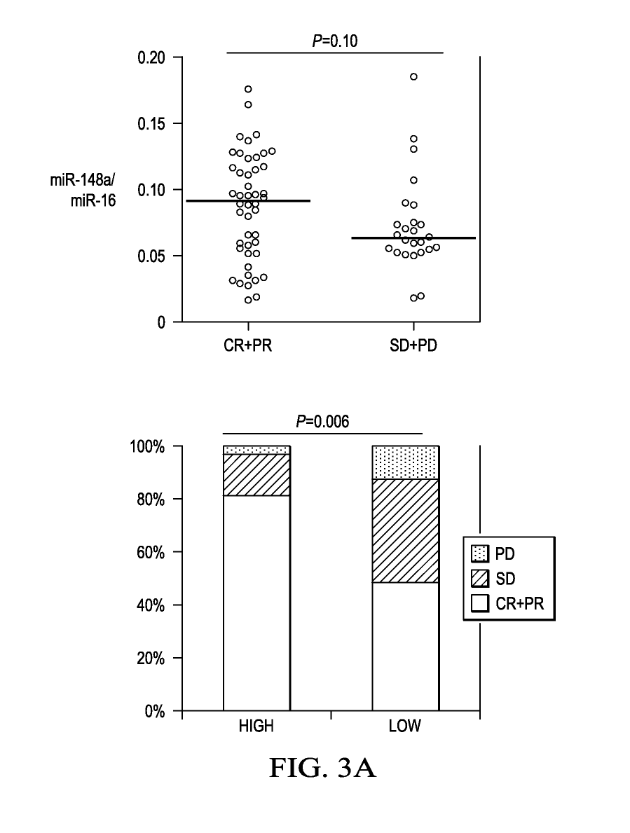

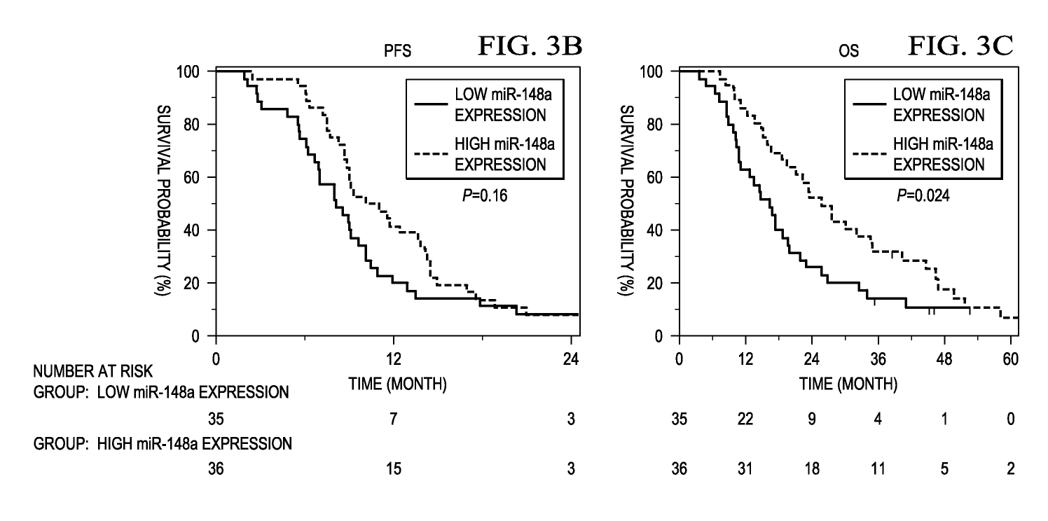

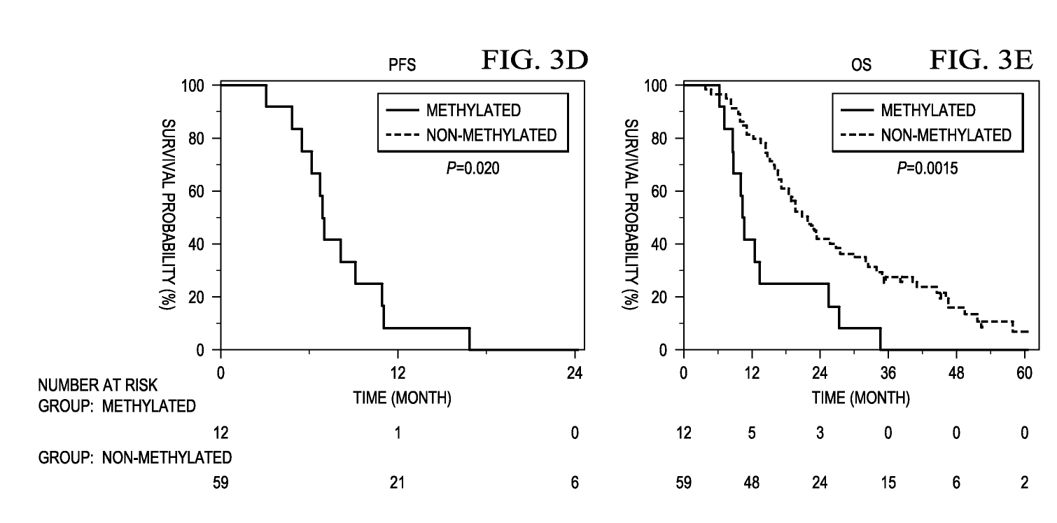

Low miR-148a expression is associated with worse a therapeutic response and worse survival in stage IV CRC. Next, the inventors elucidated whether miR-148a status had a potential for predicting therapeutic outcome in patients with stage IV CRC treated with 5-FU and oxaliplatin. Age, gender, tumor location, and performance status were not significantly different between the high and low expression groups (Table 1). Tumors from nonresponders (stable disease and progressive disease) showed a trend toward lower miR-148a expression compared with those from responders (complete response and partial response) (median, 0.063 vs. 0.092, p=0.10; FIG. 3A, left). Nonetheless, when the stage IV tumors were divided into the low and high miR-148a expression groups, the low expression group was significantly associated with an unfavorable therapeutic response (responders, 49% vs. 81%, p=0.006; FIG. 3A, right). At Kaplan-Meyer analysis, the low expression group showed a trend toward worse PFS (median, 8.1 vs. 10.1 months, p=0.16; FIG. 3B, left) and significantly worse OS (16.1 vs. 25.6 months, p=0.024; FIG. 3B, right). In addition to the expression status, miR-148a methylation status also associated with both worse PFS (methylated vs. non-methylated, 6.9 vs. 9.3 months, p=0.020; FIG. 3C, left) and OS (10.2 vs. 21.8 months, p=0.0015; FIG. 3C, right).

TABLE 3
Univariable and multivariable analysis of miR-148a expression,
methylation and overall survival in stage IV CRC patients.

| | | Univariable | | Multivariable | |
|---|---|---|---|---|---|
| | | HR | | HR | |
| | | (95% CI) | P | (95% CI) | P |
| Age, years | >60 | 1.0 | | | |
| | <60 | 0.91 | 0.71 | | |
| | | (0.55-1.50) | | | |
| Gender | Male | 1.0 | | | |
| | Female | 0.68 | 0.16 | | |
| | | (0.40-1.16) | | | |
| Tumors | Colon | 1.0 | | | |
| location | Rectum | 1.41 | 0.27 | | |
| | | (0.76-2.60) | | | |
| Performance | 0-1 | 1.0 | | | |
| status | 2 | 2.62 | 0.010^{a} | | |
| | | (1.26-5.43) | | | |
| miR-148a | High | 1.0 | | 1.0 | |
| expression | Low | 1.79 | 0.026^{a} | 1.93 | 0.014^{a} |
| | | (1.08-2.98) | | (1.15-3.23) | |
| miR-148a | No | 1.0 | | 1.0 | |
| methylation | Yes | 2.76 | 0.002^{a} | 3.04 | 0.0011^{a} |
| | | (1.44-5.28) | | (1.56-5.93) | |

^{a}P < 0.05
Abbreviation:
HR, hazard ratio;
CI, confidence interval.

The inventors also evaluated the predictive value of miR-148a in a Cox proportional-hazard model. In univariate analysis, worse PS(HR 2.62, 95% CI 1.26-5.43, p=0.010), lower miR-148a expression (HR 1.79, 95% CI 1.08-2.98, p=0.026) and miR-148a hypermethylation (HR 2.76, 95% CI 1.44-5.28, p=0.002) were significantly associated with worse survival (Table 3). In the final multivariate model that included these three factors, both miR-148a expression status (HR 1.93, 95% CI 1.15-3.23, p=0.014) and miR-148a hypermethylation (HR 3.04, 95% CI 1.56-5.93, p=0.0011) emerged as independent predictive factors that were associated with poorer outcome (Table 3).

Using a large cohort of patients with CRC, the present inventors found that miR-148a expression is dysregulated and has prognostic and predictive value in CRC. At least five major findings for the significance of involvement of miR-148a dysregulation in CRC were found. First, miR-148a is down regulated in cancer cells in advanced stage CRC. Second, the methylation status of the promoter region located approximately 500 bp upstream from the mature miR-148a sequence is inversely correlated with its expression status. Third, the low miR-148a expression is associated and correlates with poorer prognosis of patients with stage III colon cancer treated with 5-FU-based chemotherapy. Fourth, low miR-148a expression is associated and correlates with worse therapeutic response and poorer survival of patients with stage IV CRC treated with 5-FU and oxaliplatin. Fifth, miR-148a methylation status is a predictor for worse prognosis in stage IV CRC.

The present inventors recognize that miR-148a is dysregulated in several cancers including CRC. Bandres et al. demonstrated that miR-148a was up-regulated in CRC tissues using 12 CRC tissues and matched normal colonic mucosa (15). More recently, Chen et al. have reported an opposing result that miR-148a is down-regulated in cancer tissues from 101 CRC patients and its low expressions are significantly associated with increased size of tumors and advanced pT stage but not with pTNM stage (21). On the other hand, Zhang et al. have not observed significant alterations of miR-148a in 42 CRC tumors compared to adjacent normal tissues (30). These conflicting results may be attributed to the difference in the way of quantifying the expression and/or in the number of tumor analyzed. In the present study, using a large cohort of 273 patients with CRC, the present inventors have provided evidence that miR-148a is more down-regulated in advanced stage and its down-regulation is associated with higher recurrence risk in stage III patients, and with worse therapeutic response and poorer survival in stage IV disease. In particular, none of reports have demonstrated that the expression status of miR-148a or even other miRNA is associated with therapeutic response in stage IV CRC patients treated with cytotoxic chemotherapy. One of the striking findings in the present study suggests that stage IV patients having tumors with high miR-148a expression are likely to more benefit from cytotoxic chemotherapy than those with low expression, and such high expression group of patients should be treated with chemotherapy early as possible. These finding are significant for the improvement of decision-making steps in management of metastatic CRC.

Although the exact mechanisms how miR-148a down-regulation contributes to promotion of malignant potential of CRC cells and/or resistance to chemotherapy remains to be further elucidated, recent evidences in other cancers have provided some clues to help account for the effect of miR-148a alterations on cellular chemosensitivity. Fujita, et al., have reported that miR-148a directly targets MSK1 and the transfection of its precursor increases sensitivity to paclitaxel in prostate cancer cells (23). miR-148a has also been shown to improve response to cisplatin and 5-FU in esophageal cancer cells (31). In addition to these findings in vitro, Langer, et al., have reported that in young patients with acute myeloid leukemia, miR-148a expression was inversely associated with the brain and acute leukemia, cytoplasmic (BAALC) gene expression, and the higher BAALC expression was associated with worse prognosis of patients treated with chemotherapy (32). The present results demonstrate that miRNA-148a expression status is a predictive marker in stage IV CRC.

The present inventors recognize that miR-148a dysregulation may be involved in metastasis steps of CRC and other cancers and that Lujambio, et al., reveals that CRC tumors with miR-148a methylation exist more frequently in patients who eventually had recurrence than in those who did not, although the number of patients analyzed was relatively small (N=32) (20), and that ectopic expression of miR-148a resulted in suppression of tumor invasion and dissemination in vitro and in vivo (20). Zheng, et al., have recently reported that miR-148a suppresses metastasis by down-regulating ROCK1 in gastric cancers (33). The present inventors find that in a large cohort of patients, low miR-148a expression status is associated and correlates with increased risk of recurrence especially in stage III patients. For the first time, the present inventors have found that stage IV CRC patients with high miR-148a expression are more likely to benefit from cytotoxic chemotherapy, highlighting the potentially novel predictive value of this miRNA as a decision-making tool in the management of patients with CRC.

The present inventors also found methylation-mediated silencing of this miRNA by comparing between expression and methylation levels using a large number of CRC tumors. Lujambio et al. (20), Kalimutho et al. demonstrate that miR-148a was hyper-methylated in 51 out of 78 (65%) CRC (28); however, However, neither of these studies performed miR-148a expression analysis and directly correlated their results with hypermethylation in tissues. Furthermore, both studies analyzed miR-148a methylation status using a non-quantitative methylation-specific PCR method, which is notoriously nonspecific for methylation, and does not provide a threshold for methylation that correlates with transcriptional inactivation of the gene. The strength of our study is that we determined miR-148a expression by qRT-PCR, and correlated the expression data with quantitative bisulfite pyrosequencing results, which is a more robust approach for demonstrating methylation-mediated dysregulation of any gene. Accordingly, the inventors observed a significant inverse association between methylation and expression, reinforcing the concept that miR-148a down-regulation in CRC is due, in part, to promoter hypermethylation. The inventors also noted a significant and independent association between miR-148a methylation and poor survival in stage IV patients, highlighting that expression and methylation status of miR-148a might be useful as prognostic/predictive markers in CRC. Finally, the inventors confirmed RT-PCR based expression results by performing ISH on FFPE tissues, which allows a direct morphologic representation of the miRNA expression in the tissues. In these studies, the inventors observed a significant correlation between qRT-PCR and ISH data, which provides a direct translational application of ISH in clinical practice.

The present inventors conducted a retrospective analysis of a number of patients, including those with stage IV CRC. It was found that miR-148a is down-regulated through methylation-mediated silencing in advanced stage CRC and that the expression as well as the methylation status of miR-148a has predictive significance for patients treated with cytotoxic drug therapy.

Thus, this study describes the clinical significance of miR-148a in CRC, wherein it is demonstrate that its expression is frequently down-regulated, particularly in advanced stage tumors. Furthermore, this study builds upon growing evidence that miRNA expression can be epigenetically regulated. These data indicate for the first time that miR-148a expression, as well as its methylation status, may serve as predictive biomarkers in CRC. These data also validate the predictive value of miR-148a in the management of CRC patients treated with conventional chemotherapy and/or combinations of molecular-targeted drugs.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Jemal A, Siegel R, Xu J, Ward E. Cancer statistics, 2010. CA Cancer J. Clin. 2010; 60:277-300.
2. Karapetis C S, Khambata-Ford S, Jonker D J, O'Callaghan C J, Tu D, Tebbutt N C, et al. K-ras mutations and benefit from cetuximab in advanced colorectal cancer. N Engl J. Med. 2008; 359:1757-65.
3. Amado R G, Wolf M, Peeters M, Van Cutsem E, Siena S, Freeman D J, et al. Wild-type KRAS is required for panitumumab efficacy in patients with metastatic colorectal cancer. J Clin Oncol. 2008; 26:1626-34.
4. Boland C R, Goel A. Microsatellite instability in colorectal cancer. Gastroenterology. 2010; 138:2073-87 e3.
5. Ribic C M, Sargent D J, Moore M J, Thibodeau S N, French A J, Goldberg R M, et al. Tumor microsatellite-instability status as a predictor of benefit from fluorouracil-based adjuvant chemotherapy for colon cancer. N Engl J. Med. 2003; 349:247-57.
6. Sargent D J, Marsoni S, Monges G, Thibodeau S N, Labianca R, Hamilton S R, et al. Defective mismatch repair as a predictive marker for lack of efficacy of fluorouracil-based adjuvant therapy in colon cancer. J Clin Oncol. 2010; 28:3219-26.
7. Vilar E, Gruber S B. Microsatellite instability in colorectal cancer—the stable evidence. Nat Rev Clin Oncol. 2010; 7:153-62.
8. Sinicrope F A, Foster N R, Thibodeau S N, Marsoni S, Monges G, Labianca R, et al. DNA mismatch repair status and colon cancer recurrence and survival in clinical trials of 5-fluorouracil-based adjuvant therapy. J Natl Cancer Inst. 2011; 103:863-75.
9. Jover R, Nguyen T P, Perez-Carbonell L, Zapater P, Paya A, Alenda C, et al. 5-Fluorouracil adjuvant chemotherapy does not increase survival in patients with CpG island methylator phenotype colorectal cancer. Gastroenterology. 2011; 140:1174-81.
10. Van Schaeybroeck S, Allen W L, Turkington R C, Johnston P G. Implementing prognostic and predictive biomarkers in CRC clinical trials. Nat Rev Clin Oncol. 2011; 8:222-32.
11. Bohanes P, Labonte M J, Lenz H J. A review of excision repair cross-complementation group 1 in colorectal cancer. Clin Colorectal Cancer. 2011; 10:157-64.
12. Koopman M, Venderbosch S, Nagtegaal I D, van Krieken J H, Punt C J. A review on the use of molecular markers of cytotoxic therapy for colorectal cancer, what have we learned? Eur J. Cancer. 2009; 45:1935-49.
13. Calin G A, Croce C M. MicroRNA signatures in human cancers. Nat Rev Cancer. 2006; 6:857-66.
14. Garzon R, Calin G A, Croce C M. MicroRNAs in Cancer. Annu Rev Med. 2009; 60:167-79.
15. Bandres E, Cubedo E, Agirre X, Malumbres R, Zarate R, Ramirez N, et al. Identification by Real-time PCR of 13 mature microRNAs differentially expressed in colorectal cancer and non-tumoral tissues. Mol. Cancer. 2006; 5:29.
16. Toyota M, Suzuki H, Sasaki Y, Maruyama R, Imai K, Shinomura Y, et al. Epigenetic silencing of microRNA-34b/c and B-cell translocation gene 4 is associated with CpG island methylation in colorectal cancer. Cancer Res. 2008; 68:4123-32.
17. Michael M Z, SM O C, van Holst Pellekaan N G, Young G P, James R J. Reduced accumulation of specific microRNAs in colorectal neoplasia. Mol Cancer Res. 2003; 1:882-91.
18. Schetter A J, Leung S Y, Sohn J J, Zanetti K A, Bowman E D, Yanaihara N, et al. MicroRNA expression profiles associated with prognosis and therapeutic outcome in colon adenocarcinoma. JAMA. 2008; 299:425-36.
19. Schepeler T, Reinert J T, Ostenfeld M S, Christensen L L, Silahtaroglu A N, Dyrskjot L, et al. Diagnostic and prognostic microRNAs in stage II colon cancer. Cancer Res. 2008; 68:6416-24.
20. Lujambio A, Calin G A, Villanueva A, Ropero S, Sanchez-Cespedes M, Blanco D, et al. A microRNA DNA methylation signature for human cancer metastasis. Proc Natl Acad Sci USA. 2008; 105:13556-61.
21. Chen Y, Song Y, Wang Z, Yue Z, Xu H, Xing C, et al. Altered expression of MiR-148a and MiR-152 in gastrointestinal cancers and its clinical significance. J Gastrointest Surg. 2010; 14:1170-9.
22. Takagi S, Nakajima M, Mohri T, Yokoi T. Post-transcriptional regulation of human pregnane X receptor by micro-RNA affects the expression of cytochrome P450 3A4. J Biol. Chem. 2008; 283:9674-80.
23. Fujita Y, Kojima K, Ohhashi R, Hamada N, Nozawa Y, Kitamoto A, et al. MiR-148a attenuates paclitaxel resistance of hormone-refractory, drug-resistant prostate cancer PC3 cells by regulating MSK1 expression. J Biol. Chem. 2010; 285:19076-84.
24. Liffers S T, Munding J B, Vogt M, Kuhlmann J D, Verdoodt B, Nambiar S, et al. MicroRNA-148a is down-regulated in human pancreatic ductal adenocarcinomas and regulates cell survival by targeting CDC25B. Lab Invest. 2011; 91:1472-9.
25. Pan W, Zhu S, Yuan M, Cui H, Wang L, Luo X, et al. MicroRNA-21 and microRNA-148a contribute to DNA hypomethylation in lupus CD4+ T cells by directly and indirectly targeting DNA methyltransferase 1. J. Immunol. 2010; 184:6773-81.
26. Duursma A M, Kedde M, Schrier M, le Sage C, Agami R. miR-148 targets human DNMT3b protein coding region. RNA. 2008; 14:872-7.
27. Therasse P, Arbuck S G, Eisenhauer E A, Wanders J, Kaplan R S, Rubinstein L, et al. New guidelines to evaluate the response to treatment in solid tumors. European Organization for Research and Treatment of Cancer, National Cancer Institute of the United States, National Cancer Institute of Canada. J Natl Cancer Inst. 2000; 92:205-16.
28. Kalimutho M, Di Cecilia S, Del Vecchio Blanco G, Roviello F, Sileri P, Cretella M, et al. Epigenetically silenced miR-34b/c as a novel faecal-based screening marker for colorectal cancer. Br J. Cancer. 2011; 104:1770-8.
29. Lehmann U, Hasemeier B, Christgen M, Muller M, Romermann D, Langer F, et al. Epigenetic inactivation of microRNA gene hsa-mir-9-1 in human breast cancer. J. Pathol. 2008; 214:17-24.
30. Zhang H, Li Y, Huang Q, Ren X, Hu H, Sheng H, et al. MiR-148a promotes apoptosis by targeting Bcl-2 in colorectal cancer. Cell Death Differ. 2011.
31. Hummel R, Watson D I, Smith C, Kist J, Michael M Z, Haier J, et al. Mir-148a improves response to chemotherapy in sensitive and resistant oesophageal adenocarcinoma and squamous cell carcinoma cells. J Gastrointest Surg. 2011; 15:429-38.
32. Langer C, Radmacher M D, Ruppert A S, Whitman S P, Paschka P, Mrozek K, et al. High BAALC expression associates with other molecular prognostic markers, poor outcome, and a distinct gene-expression signature in cytogenetically normal patients younger than 60 years with acute myeloid leukemia: a Cancer and Leukemia Group B (CALGB) study. Blood. 2008; 111:5371-9.
33. Zheng B, Liang L, Wang C, Huang S, Cao X, Zha R, et al. MicroRNA-148a Suppresses Tumor Cell Invasion and Metastasis by Downregulating ROCK1 in Gastric Cancer. Clin Cancer Res. 2011.

What is claimed is:

1. A method to diagnose a stage of cancer of a patient suspected of having colorectal cancer comprising:
obtaining a sample from the patient suspected of having colorectal cancer;
determining a level of methylation of a miR-148a promoter or the level of expression of miR-148a; and
diagnosing a stage of colorectal cancer if the level of methylation of the miR-148a promoter is lower than in normal colonic tissue or the level of expression of miR-148a is higher than in normal colonic tissue.

2. The method of claim 1, wherein the level of expression of miR-148a of a stage III or State IV tumor is significantly lower than those of normal colonic mucosa.

3. The method of claim 1, wherein the step of determining the level of expression of miR-148a comprises the additional step of normalizing expression of miR-148a with expression of miR-16.

4. The method of claim 1, wherein the one or more samples are selected from the group consisting of a cancer biopsy, a tissue sample, a liver biopsy, a fecal sample, a cell homogenate, a blood, a serum, a plasma, one or more biological fluids, or any combinations thereof.

5. The method of claim 1, wherein the one or more samples comprise a cancer sample, a colorectal cancer sample, a control sample, or combinations thereof.

6. The method of claim 1, wherein further comprising the step of predicting a response to a cancer treatment by predicting if the patient will benefit from cytotoxic chemotherapy, wherein if level of expression of miR-148a is greater than the level in a non-cancerous sample of colorectal tissue obtained from the patient the patient will benefit from cytotoxic chemotherapy.

7. The method of claim 6, wherein a low level of expression of miR-148a indicates at least one of reduced disease-free survival, progression-free survival (PFS), or overall survival (OS), of the patient if treated by cytotoxic chemotherapy cancer treatment.

8. The method of claim 6, wherein a low level of expression of miR-148a, indicates a reduced disease-free survival of the patient suspected of having stage II and III colon cancer if treated with a thymidylate synthase inhibitor, 5-fluorouracil (5-FU) or analogs thereof, or if the patient suspected of having stage IV colon cancer if treated with 5-FU and oxaliplatin-based chemotherapy.

9. A method to manage a treatment of a patient suspected of having a colorectal cancer comprising:
obtaining one or more samples of the patient;
determining a level of expression of miR-148a; and
predicting a response to a cytotoxic chemotherapy cancer treatment, wherein an increase in the level of expression of miR-148a is predictive of an increased responsiveness to the cytotoxic chemotherapy.

10. The method of claim 9, wherein predicting a response to a cancer treatment comprises predicting that the patient will not benefit from cytotoxic chemotherapy if level of expression of miR-148a is less than the level in a normal sample.

11. The method of claim 9, wherein a low level of expression of miR-148a indicates at least one of reduced disease-free survival, progression-free survival (PFS), or overall survival (OS), of the patient if treated by cytotoxic chemotherapy cancer treatment.

12. The method of claim 9, wherein a low level of expression of miR-148a, indicates a reduced disease-free survival of the patient suspected of having stage II and III colon cancer if treated with a thymidylate synthase inhibitor or 5-fluorouracil (5-FU) or analogs thereof.

13. The method of claim 9, wherein a low expression of miR-148a, indicates a reduced disease-free survival of the patient suspected of having stage IV colon cancer if treated with 5-FU and oxaliplatin-based chemotherapy.

14. The method of claim 9, wherein determining the level of expression of miR-148a further comprises the step of normalizing expression of miR-148a with expression of miR-16.

15. The method of claim 9, wherein the one or more samples are selected from the group consisting of a cancer biopsy, a colorectal cancer tissue, a tissue sample, a liver biopsy, a colon biopsy, a rectal biopsy, a fecal sample, a cell homogenate, a blood, a serum, a plasma, one or more biological fluids, or any combinations thereof.

16. The method of claim 9, wherein predicting a response to a cancer treatment comprises predicting disease-free survival (DFS), progression-free survival (PFS), overall survival (OS), or combinations thereof.

17. The method of claim 9, wherein predicting a response to a cancer treatment comprises predicting a higher colorectal metastatic stage if expression of miR-148a is above the median for miR-148a expression in a normal tissue.

18. The method of claim 9, wherein an increase in miR-148a expression or a decrease in miR-148a promoter methylation indicates that the colorectal cancer is stage II, stage III, or stage IV.

19. The method of claim 9, further comprising indicating cytotoxic chemotherapy if the level of expression of miR-148a is above the median for miR-148a expression in a normal tissue.

20. The method of claim 9, further comprising the step of contraindicating cytotoxic chemotherapy if the level of expression of miR-148a is below the median for miR-148a expression in a normal tissue.

21. A method for selecting a cancer therapy for a patient diagnosed with metastatic colorectal cancer comprising the steps of:
determining a level of expression of miR-148a in one or more biological samples of the patient;
selecting a first or second cancer therapy based on the level of expression of miR-148a; and
treating the patient with a first cancer therapy comprising anti-growth hormone or anti-hormone receptor therapy or treating the patient with a second cancer therapy comprising cytotoxic chemotherapy.

22. The method of claim 21, wherein the anti-growth hormone comprises a VEGF antagonist, an anti-VEGF antibody, bevacizumab.

23. The method of claim 21, wherein the anti-growth hormone receptor comprises an EGFR antagonist, an anti-EGFR antibody, cetuximab, or panitumumab.

24. The method of claim 21, wherein determining miR-148a activity comprises comparing level of expression of miR-148a with a level of expression of a control.

25. The method of claim 21, wherein the one or more samples are selected from the group consisting of a cancer biopsy, a tissue sample, a liver biopsy, a fecal sample, a cell homogenate, a blood, a serum, a plasma, one or more biological fluids, or any combinations thereof.

26. The method of claim 21, wherein the one or more samples comprise a colorectal cancer sample, a control sample, or combinations thereof.

27. The method of claim 21, wherein selecting survival of the patient comprises selecting cytotoxic chemotherapy if miR-148a activity is high.

28. A method to predict survival of a patient suspected of having stage III or stage IV colorectal cancer comprising:
obtaining one or more biological samples of the patient;
determining a level of expression of miR-148a; and
predicting survival probability of the patient, wherein an increase in the level of expression of miR-148a is predictive of an increased responsiveness to the cytotoxic chemotherapy.

29. The method of claim 28, wherein the colorectal cancer is stage II or stage III and predicting survival probability comprises predicting a 5-year disease-free survival of less than 54% if the level of expression of miR-148a is below 0.69-fold of a level of expression of miR-148a of normal mucosa.

30. The method of claim 28, wherein the chemotherapy comprises treatment with 5-fluorouracil or a combination of Folinic Acid (FOL), Fluorouracil (5-FU) and Oxaliplatin (OX), or irinotecan.

31. A method of performing a clinical trial to evaluate a candidate drug believed to be useful in treating colorectal cancer, the method comprising:
(a) determining a level of miR-148a expression in one or more biological sample of the patient;
(b) administering a candidate drug to a first subset of patients, and
a placebo to a second subset of patients;
a comparable drug to a second subset of patients; or
a drug combination of the candidate drug and another active agent to a second subset of patients;
(c) repeating step (a) after the administration of the candidate drug or the placebo, the comparable drug or the drug combination; and
(d) monitoring a change in the level of miR-148a expression of the first subset of patients as compared to the second subset of patients, wherein a statistically significant increase indicates that the candidate drug is useful in treating colorectal cancer.

32. A method to diagnose a stage of cancer of a patient suspected of having colorectal cancer comprising:
obtaining a sample of the patient suspected of having colorectal cancer;
determining a level of expression of miR-148a; and
diagnosing a stage of colorectal cancer, wherein the level of expression of miR-148a of stage III and IV tumors is significantly lower than those of normal colonic mucosa.

33. A method for selecting a cancer therapy for a patient diagnosed with metastatic colorectal cancer comprising the steps of:
determining a level of methylation of a miR-148a promoter in one or more biological samples of the patient;
selecting the cancer therapy based on the determination of the level of methylation of the miR-148a promoter; and
treating the patient with a first treatment comprising an anti-growth hormone or anti-hormone receptor therapy if the patient does not have decreased methylation of the miR-148a promoter; or treating the patient with a second treatment comprising cytotoxic chemotherapy if the patient has decreased methylation of the miR-148a promoter.

34. A method to predict survival of a patient suspected of having colorectal cancer comprising:
obtaining one or more biological samples of the patient;
determining a level of methylation of a miR-148a promoter; and
predicting survival probability of the patient.

35. A method of performing a clinical trial to evaluate a candidate drug believed to be useful in treating colorectal cancer, the method comprising:
(a) determining a level of methylation of a miR-148a promoter in one or more biological samples of patients;
(b) administering a candidate drug to a first subset of patients, and
a placebo to a second subset of patients;
a comparable drug to a second subset of patients; or a drug combination of the candidate drug and another active agent to a second subset of patients;
(c) repeating step (a) after the administration of the candidate drug or the placebo, the comparable drug or the drug combination; and
d) monitoring a change in the level of methylation of the miR-148a promoter of the first subset of patients as compared to the second subset of patients, wherein a statistically significant reduction indicates that the candidate drug is useful in treating colorectal cancer.

36. A kit for determining the stage of colorectal cancer in a human subject comprising:
a biomarker detecting reagent for measuring level of methylation of a miR-148a promoter or the level of expression of miR-148a in a sample obtained from the human subject; and
instructions for the use of the biomarker detecting reagent in determining the stage of colorectal cancer, wherein the instructions comprise providing step-by-step directions to compare the level of methylation of the miR-148a promoter or the level of expression of miR-148a from the sample, wherein a decrease in the methylation of the miR-148a promoter or an increase in expression of miR-148a in the sample versus a normal colonic tissue is indicative of a higher stage of colorectal cancer.

37. The kit of claim 36, wherein the level of methylation of the miR-148a promoter is determined by quantitative bisulfite pyrosequencing, thin layer chromatography (TLC), high performance liquid chromatography (HPLC), mass spectrometry (MS), nanopore amperometry, nanopore sequencing, single-molecule, real-time (SM-RT) sequencing, endonuclease digestion, microarrays, matrix-assisted laser desorption ionization time-of-flight (MALDI-TOF) mass spectrometry, and next-generation sequencing.

38. The kit of claim 36, wherein the biological samples are selected from the group consisting of a tissue sample, a plasma sample, a fecal sample, a cell homogenate, a blood sample, one or more biological fluids, or any combinations thereof.

39. The kit of claim 36, wherein the level of expression of miR-148a from the sample is determined by nanostring, microarray expression profiling, PCR, reverse transcriptase PCR, reverse transcriptase real-time PCR, quantitative real-time PCR, end-point PCR, multiplex end-point PCR, cold PCR, ice-cold PCR, mass spectrometry, or nucleic acid sequencing.

40. The kit of claim 36, wherein a low level of expression of miR-148a indicates at least one of reduced disease-free survival, progression-free survival (PFS), or overall survival (OS), of the patient if treated by cytotoxic chemotherapy cancer treatment.
